(19) United States
(12) Patent Application Publication
(10) Pub. No.: US 2022/0033355 A1
(43) Pub. Date: Feb. 3, 2022

(54) CRYSTAL FORM A OF 2-(2, 5-DIOXOPYRROLIDIN-1YL) ETHYL METHYL FUMARATE, PREPARATION METHOD THEREFOR AND USE THEREOF

(71) Applicant: SHENZHEN RENTAI PHARMATECH LTD., Shenzhen (CN)

(72) Inventors: Guobin REN, Shenzhen (CN); Dongxu YI, Shenzhen (CN); Weijie JI, Shenzhen (CN); Jiajun HUANG, Shenzhen (CN)

(21) Appl. No.: 17/487,106

(22) Filed: Sep. 28, 2021

Related U.S. Application Data
(63) Continuation of application No. PCT/CN2019/080420, filed on Mar. 29, 2019.

(51) Int. Cl. C07D 207/404

(52) U.S. Cl. CPC C07D 207/404; C07B 2200/13

(57) ABSTRACT

A crystal form A of 2-(2, 5-dioxopyrrolidin-1yl)ethyl methyl fumarate has a good light irradiation stability, high-temperature stability and high-humidity stability.

### RELATED APPLICATIONS

This application is a continuation application of International Patent Application No. PCT/CN2019/080420 with an international filing date of Mar. 29, 2019, designating the United States, the disclosure of which is hereby incorporated by reference in its entirety.

### FIELD OF THE DISCLOSURE

The present application relates to the technical field of crystal form preparation, in particular to a crystal form A of 2-(2, 5-dioxopyrrolidin-1yl)ethyl methyl fumarate, preparation method therefor and use thereof.

### BACKGROUND

2-(2, 5-dioxopyrrolidin-1yl)ethyl methyl fumarate of below formula (I), also known as ALKS8700, is a prodrug of fumarates developed by Alkermes Pharma Ireland Ltd (i.e. Alkermes). This compound is known as a drug for treating psoriasis and multiple sclerosis (CN105452213B and CN107205985B). Fumarates are approved in Germany for the treatment of psoriasis, are being evaluated in the United States for the treatment of psoriasis and multiple sclerosis, and have been proposed for use in treating a wide range of immunological, autoimmune, and inflammatory diseases and conditions. However, fumarates still have many disadvantages in use, such as side effects including gastrointestinal reactions, and repeated administration. ALKS8700 can improve times of administration of fumarate and reduce the side effects of administration, so it is of great significance to study ALKS8700.

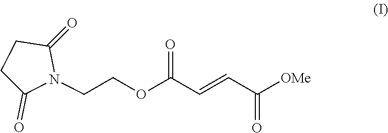 [SMILES: COC(=O)/C=C/C(=O)OCCN1C(=O)CCC1=O]

CN105452213B discloses a preparation method of the compound of formula (I). By referring to the following scheme, the preparation method comprises: (1) adding monomethyl fumarate (MMF) and benzotriazole-N,N,N′,N′-tetramethyluronium hexafluorophosphate (HBTU) into diisopropylethylamine and stirring, adding ethanol and water successively, and extracting in ethyl acetate, combining the organic layers and washing with water, then drying, purifying by grinding with diethyl ether, to prepare the compound of formula (I).

The inventors of the present application prepared the compound of formula (I) according to the preparation method as described in CN105452213B, and carried out analysis. The results showed that the prepared compound of formula (I) would undergo crystal transition in environment of high temperature or high humidity or under lights, and the resulting compound is unstable in physical state, thus cannot be used as a bulk pharmaceutical chemical. Therefore, a strict management would be required during use of the compound, rendering the compound unsuitable for use as a bulk pharmaceutical chemical.

### SUMMARY

Accordingly, it is an object of the present application to provide a crystal form A of 2-(2, 5-dioxopyrrolidin-1yl)ethyl methyl fumarate of formula (I), a preparation method therefor and use thereof. The crystal A has significantly improved stability and has a residual solvent content significantly reduced to below 0.01%.

In one aspect, the present application provides a crystal form A of 2-(2, 5-dioxopyrrolidin-1yl)ethyl methyl fumarate, wherein the X-ray powder diffraction thereof using Cu-Kα radiation has characteristic peaks at 2θ diffraction angles of 13.5±0.2°, 17.9±0.2°, 23.0±0.2° and 27.3±0.2°.

In some embodiments, the X-ray powder diffraction thereof using Cu-Kα radiation has further characteristic peaks at 2θ diffraction angles of 13.3±0.2°, and 18.2±0.2°.

In some embodiments, the X-ray powder diffraction thereof using Cu-Kα radiation has further characteristic peaks at 2θ diffraction angles of 19.3±0.2°, and 19.6±0.2°.

In some embodiments, the X-ray powder diffraction thereof using Cu-Kα radiation has further characteristic peaks at 2θ diffraction angles of 16.6±0.2°, 20.9±0.2°, 22.0±0.2°, 24.3±0.2°, 25.3±0.2°, and 30.6±0.2°.

In some embodiments, the X-ray powder diffraction thereof using Cu-Kα radiation has further characteristic peaks at 2θ diffraction angles of 6.92±0.2°, 11.5±0.2°, 16.1±0.2°, 23.7±0.2°, 26.9±0.2°, and 31.1±0.2°.

In some embodiments, the crystal form A has following characteristic peaks in X-ray powder diffraction pattern:

| No. of Peaks | 2θ (°) | I % |
|---|---|---|
| 1 | 13.3 | 72.3 |
| 2 | 13.54 | 19.8 |
| 3 | 17.878 | 82.2 |
| 4 | 22.962 | 100 |
| 5 | 27.34 | 97.2 |

In some embodiments, the crystal form A has following characteristic peaks in X-ray powder diffraction pattern:

| No. of Peaks | 2θ (°) | I % |
|---|---|---|
| 1 | 6.92 | 12.8 |
| 2 | 11.481 | 6.1 |
| 3 | 13.3 | 72.3 |
| 4 | 13.54 | 19.8 |
| 5 | 16.562 | 14.5 |
| 6 | 17.878 | 82.2 |
| 7 | 18.24 | 5.1 |
| 8 | 19.282 | 2.5 |
| 9 | 19.644 | 2.9 |
| 10 | 20.901 | 53 |
| 11 | 21.98 | 18.4 |
| 12 | 22.962 | 100 |
| 13 | 24.26 | 25.7 |
| 14 | 26.858 | 13.5 |
| 15 | 27.34 | 97.2 |

In some embodiments, the crystal form A has following characteristic peaks in X-ray powder diffraction pattern:

| No. of Peaks | 2-Theta | I % | d (Å) |
|---|---|---|---|
| 1 | 6.92 | 12.8 | 12.764 |
| 2 | 11.481 | 6.1 | 7.7013 |
| 3 | 13.3 | 72.3 | 6.6517 |
| 4 | 13.54 | 19.8 | 6.5344 |
| 5 | 16.562 | 14.5 | 5.3481 |
| 6 | 17.878 | 82.2 | 4.9572 |
| 7 | 18.24 | 5.1 | 4.8597 |
| 8 | 19.282 | 2.5 | 4.5995 |
| 9 | 19.644 | 2.9 | 4.5155 |
| 10 | 20.901 | 53 | 4.2467 |
| 11 | 21.98 | 18.4 | 4.0405 |
| 12 | 22.962 | 100 | 3.87 |
| 13 | 23.681 | 7.3 | 3.754 |
| 14 | 24.26 | 25.7 | 3.6657 |
| 15 | 25.34 | 8.2 | 3.5119 |
| 16 | 26.858 | 13.5 | 3.3167 |
| 17 | 27.34 | 97.2 | 3.2593 |
| 18 | 30.621 | 14.4 | 2.9171 |
| 19 | 31.099 | 11.6 | 2.8734 |

In some embodiments, the crystal form A has an X-ray powder refraction pattern substantially as shown in FIG. 4.

In some embodiments, the crystal form A has a characteristic endothermic peak in a temperature range of 96.0° C.-107.0° C. measured by differential scanning calorimetry.

In some embodiments, the crystal form A has a differential scanning calorimetry curve substantially as shown in FIG. 5.

In some embodiments, the crystal form A has a weight loss of less than 0.01% before a temperature of 125° C. in its thermo gravimetric analysis curve.

In some embodiments, the crystal form A has a thermo gravimetric analysis curve substantially as shown in FIG. 6.

In another aspect, the present application further provides a method for preparing the crystal form A, comprising the following steps of: dissolving 2-(2, 5-dioxopyrrolidin-1yl)ethyl methyl fumarate by adding a good solvent thereto, evaporating the solvent or cooling to give a solid, and drying the solid to obtain the crystal form A as a powder.

In some embodiments, said dissolving is performed by adding the good solvent at a temperature of 50° C. to 65° C., and said cooling is performed at a temperature of −18° C. to 4° C. to give a solid.

In another aspect, the present application further provides a method for preparing the crystal form A, comprising the following steps of: dissolving 2-(2, 5-dioxopyrrolidin-1yl)ethyl methyl fumarate by adding a good solvent, thereto, then adding a poor solvent, separating a solid and drying to obtain the crystal form A as a powder.

In some embodiments, the good solvent is added at a temperature of 15° C. to 35° C. for dissolving, and the poor solvent is added at a temperature of 15° C. to 35° C. to obtain a solid.

In some embodiments, the good solvent is an organic solvent selected from the group consisting of a lower alcohol, a lower ketone, a lower ester, a lower nitrile, and a lower ether; preferably, the lower alcohol is selected from the group consisting of methanol, ethanol, isopropanol or n-butanol, the lower ketone is acetone or 4-methyl-2-pentanone, the lower ester is ethyl acetate, the lower ether is tetrahydrofuran or dioxane, and the lower nitrile is acetonitrile.

In some embodiments, a ratio of 2-(2, 5-dioxopyrrolidin-1yl)ethyl methyl fumarate to the good solvent is (10-40) mg:(0.1-5) mL.

In some embodiments, the poor solvent is select from n-heptane, n-hexane, or absolute ethyl ether.

In another aspect, the present application further provides a pharmaceutical composition, comprising the crystal form A and a pharmaceutically acceptable excipient.

In another aspect, the present application further provides use of a pharmaceutically effective amount of the crystal form A, or of the crystal form A prepared by the method, or of the pharmaceutical composition, in the manufacture of a medicament for treating a neurological diseases.

In some embodiments, the neurological disease is multiple sclerosis or psoriasis.

In some embodiments, the medicament is administered orally, parenterally, intradermally, intrathecally, intramuscularly, subcutaneously, vaginally, as a buccal, sublingually, rectally, as a topical, inhalation, intranasal, or transdermally.

In another aspect, the present application further provides a method for treating multiple sclerosis or psoriasis, comprising the step of administering a pharmaceutically effective amount of the pharmaceutical composition to a patient.

The technical solutions of the present application have the following advantages:

1. The crystal form A of the compound of formula (I) provided in the present application has high purity, and good solubility in water, buffer solution or organic solvent, which is beneficial to prepare a medicament.

2. The crystal form A of the compound of formula (I) provided in the present application has good light stability, high temperature stability, and high humidity stability, and has a moisture content or other solvent content as low as 0.002563%. When the relative humidity is increased from 0 to RH 90%, the crystal form A show a weight increase by hygroscopicity of not higher than 0.35%, indicating the moisture absorption by the crystal form A is slow. The crystal form A can be prepared with a simple preparation process under mild conditions, and the quality is stable, all of which facilitate large-scale industrial production.

3. The crystal form A of the compound of formula (I) provided by the present application has an improved powder flowability when compared with crystal form I, and is suitable to prepare formulations with stable active ingredient content.

4. The crystal form A of the compound of formula (I) provided by the present application has better efficacy in animal body, longer half-life period and higher exposure when compared with crystal form I.

### BRIEF DESCRIPTION OF THE DRAWINGS

In order to more clearly describe the specific embodiments of the present application or the technical solutions in the prior art, drawings used in the specific embodiments or the description of the prior art will be briefly introduced as follows. Obviously, the drawings in the following description are some embodiments of the present application, and other drawings can be obtained according to these drawings without paying creative labor for those skilled in the art.

FIG. 1 shows an X-ray powder diffraction (XRPD) pattern of crystal form I in Example 1 of the present application;

FIG. 2 shows a differential scanning calorimetry (DSC) curve of crystal form I in Example 1 of the present application;

FIG. 3 shows a thermo gravimetric analysis (TGA) curve of crystal form I in Example 1 of the present application;

FIG. 4 shows an XRPD pattern (peak positions are marked) of crystal form A in Example 2 of the present application;

FIG. 5 shows a DSC curve of crystal form A in Example 2 of the present application;

FIG. 6 shows a TGA curve of crystal form A in Example 2 of the present application;

FIG. 7 shows an XRPD pattern of crystal form A in Example 3 of the present application;

FIG. 8 shows an XRPD pattern of crystal form A in Example 4 of the present application;

FIG. 9 shows an XRPD pattern of crystal form A in Example 5 of the present application;

FIG. 10 shows an XRPD pattern of crystal form A in Example 6 of the present application;

FIG. 11 shows an XRPD pattern of crystal form A in Example 7 of the present application;

FIG. 12 shows an XRPD pattern of crystal form A in Example 8 of the present application;

FIG. 13 shows a dynamic vapor sorption (DVS) curve of crystal form I in Example 1 of the present application;

FIG. 14 shows a DVS curve of crystal form A in Example 2 of the present application;

FIG. 15 shows comparison of XRPD patterns of crystal form A of the present application to study stability under lights;

FIG. 16 shows comparison of XRPD patterns of crystal form A of the present application to study stability under high temperatures;

FIG. 17 shows comparison of XRPD patterns of crystal form A of the present application to study stability under high humidity 1;

FIG. 18 shows comparison of XRPD patterns of crystal form A of the present application to study stability under high humidity 2;

FIG. 19 shows comparison of XRPD patterns of crystal form I in Experimental Example 4 of the present application to study stability under lights;

FIG. 20 shows comparison of XRPD patterns of crystal form I in Experimental Example 4 of the present application to study stability under temperatures;

FIG. 21 shows comparison of XRPD patterns of crystal form I in Experimental Example 4 of the present application to study stability under high humidity 1;

FIG. 22 shows comparison of XRPD patterns of crystal form I in Experimental Example 4 of the present application to study stability under high humidity 2.

Corresponding reference numerals are used to indicate corresponding parts in the drawings.

### DETAILED DESCRIPTION

The embodiments of the present disclosure described below are not intended to be exhaustive or to limit the disclosure to the precise forms disclosed in the following detailed description. Rather, the embodiments are chosen and described so that others skilled in the art may appreciate and understand the principles and practices of the present disclosure.

The term “bulk pharmaceutical chemical” used in the following embodiments of the present application refers to 2-(2, 5-dioxopyrrolidin-1yl)ethyl methyl fumarate, with a chemical purity of greater than 98%, provided by Shanghai Haoyuan Biomedical Technology Co., Ltd.

Following experimental equipment and test conditions are used in the present application:

### X-Ray Powder Diffractometer XRPD

### Model: Uitima IV (Rigaku, Japan)

Method: Cu target Ka, voltage: 40 KV, current: 40 mA, test angle: 3-45°, scanning step: 0.02, exposure time: 0.2S, slit width of light pipe: 2 mm, Dtex detector.

### X-Ray Single Crystal Diffractometer SXRD

### Model: BRUKER D8 QUEST (BRUKER, Germany)

Method: Cu target, voltage: 40 KV, current: 30 mA

### Differential Scanning Calorimeter DSC

### Model: TA 2000 (TA Instruments, US)

Method: heating at a rate of 10° C./min.

### Thermal Gravimetric Analysis TGA

### Model: TA 500 (TA Instruments, US);

Method: heating at a rate of 10° C./min.

### Dynamic Vapor Sorption DVS

Model: DVS intrinsic (SMS, British);
Method: 25° C., relative humidity is stepped up at a rate of 10%, the judgment standard is change in moisture content is <0.02% over a 10-minute period.

### Light Incubator

### Model: TES-1330A (TES Electronic Corp.)

### Ultrasound Equipment

### Model: SK8200LHC (Shanghai Kedao Ultrasonic Instrument Co., Ltd.)

### Programmable Temperature and Humidity Chamber for Drug Stability

### Model: CMA-100C (Shanghai Puhan Precision Equipment Co., Ltd.)

### Example 1 Crystal Characterization of Bulk Pharmaceutical Chemical

2-(2, 5-dioxopyrrolidin-1yl)ethyl methyl fumarate, with a chemical purity of greater than 98%, purchased from and prepared by Shanghai Haoyuan Biomedical Technology Co., Ltd. according to the method disclosed in CN105452213B, is used as bulk pharmaceutical chemical.

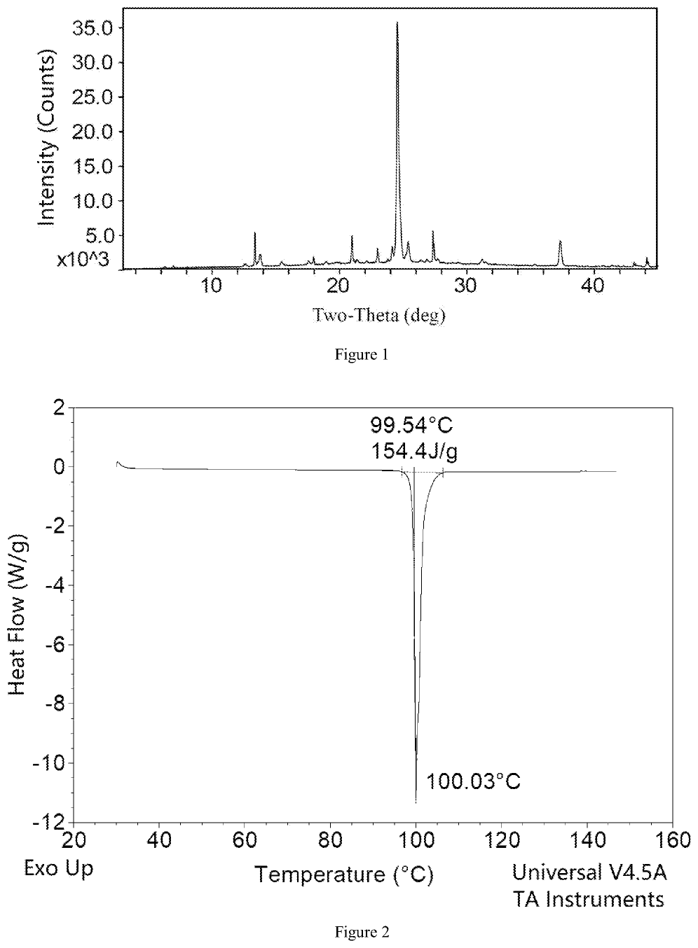

XRPD pattern for the bulk pharmaceutical chemical is measured and shown in FIG. 1. The characteristic peaks are listed in the below table. It is confirmed that the compound prepared by the method disclosed in CN105452213B is a crystal, marked as crystal form I.

TABLE 1
Characteristic peaks of crystal form I

| 2-Theta | d (Å) | I % |
|---|---|---|
| 6.262 | 14.1017 | 1.1 |
| 6.961 | 12.6876 | 0.6 |
| 12.563 | 7.0403 | 1.1 |
| 13.358 | 6.623 | 5.5 |
| 13.797 | 6.4133 | 5.9 |
| 15.459 | 5.7272 | 1.8 |
| 17.579 | 5.0409 | 1.8 |
| 17.963 | 4.934 | 2.2 |
| 18.959 | 4.677 | 1 |
| 19.405 | 4.5704 | 0.6 |
| 19.797 | 4.4808 | 1 |
| 19.962 | 4.4441 | 1 |
| 20.98 | 4.2309 | 6.3 |
| 21.362 | 4.1561 | 1.4 |
| 22.124 | 4.0145 | 0.4 |
| 22.982 | 3.8666 | 3.2 |
| 24.123 | 3.6862 | 4 |
| 24.541 | 3.6244 | 100 |
| 25.398 | 3.5039 | 5.5 |
| 26.38 | 3.3757 | 0.5 |
| 26.842 | 3.3186 | 0.7 |
| 27.347 | 3.2586 | 10 |
| 27.702 | 3.2176 | 2.4 |
| 28.938 | 3.0829 | 0.8 |
| 29.361 | 3.0394 | 0.8 |
| 30.361 | 2.9415 | 0.2 |
| 31.163 | 2.8677 | 2.8 |
| 31.441 | 2.8429 | 1.3 |
| 33.669 | 2.6598 | 0.3 |
| 34.603 | 2.59 | 0.3 |
| 35.301 | 2.5404 | 0.3 |
| 37.321 | 2.4074 | 11.1 |
| 39.375 | 2.2864 | 0.1 |
| 40.694 | 2.2153 | 0.2 |
| 41.364 | 2.181 | 0.3 |
| 41.857 | 2.1564 | 0.5 |
| 43.121 | 2.0961 | 1 |
| 44.124 | 2.0508 | 1.8 |

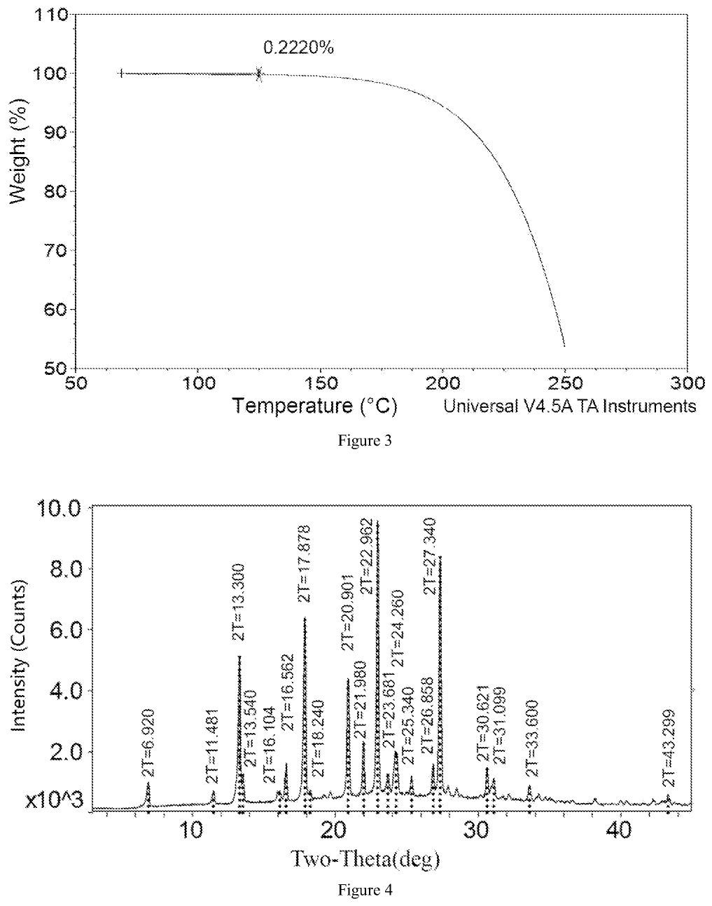

DSC and TGA curves for the bulk pharmaceutical chemical are measured and shown in FIGS. 2 and 3. It is observed that, the crystal form I has one characteristic endothermic peak at 94.0-107.0° C. in the DSC curve, and a weight loss of 0.2220% before 125° C. in the TGA curve.

### Example 2 Preparation of Crystal Form A

10.8 mg of the bulk pharmaceutical chemical is weighed out and placed into a sample bottle, and 0.4 mL of methanol is added thereto for dissolving to produce a clear solution. The clear solution is allowed to undergo slow evaporation to give a solid. The solid is vacuum dried at room temperature to obtain a white powder. XRPD pattern is measured and shown in FIG. 4. Characteristic peaks are listed in the table below.

TABLE 2
Characteristic peaks of crystal form
A

| No. of Peaks | 2-Theta | d (Å) | I % |
|---|---|---|---|
| 1 | 6.92 | 12.764 | 12.8 |
| 2 | 11.481 | 7.7013 | 6.1 |
| 3 | 13.3 | 6.6517 | 72.3 |
| 4 | 13.54 | 6.5344 | 19.8 |
| 5 | 16.104 | 5.4992 | 7.8 |
| 6 | 16.562 | 5.3481 | 14.5 |
| 7 | 17.878 | 4.9572 | 82.2 |
| 8 | 18.24 | 4.8597 | 5.1 |
| 9 | 19.282 | 4.5995 | 2.5 |
| 10 | 19.644 | 4.5155 | 2.9 |
| 11 | 20.901 | 4.2467 | 53 |
| 12 | 21.98 | 4.0405 | 18.4 |
| 13 | 22.962 | 3.87 | 100 |
| 14 | 23.681 | 3.754 | 7.3 |
| 15 | 24.26 | 3.6657 | 25.7 |
| 16 | 24.968 | 3.5634 | 1.4 |
| 17 | 25.34 | 3.5119 | 8.2 |
| 18 | 26.858 | 3.3167 | 13.5 |
| 19 | 27.34 | 3.2593 | 97.2 |
| 20 | 27.898 | 3.1954 | 1.5 |
| 21 | 28.5 | 3.1293 | 3.2 |
| 22 | 28.83 | 3.0942 | 0.3 |
| 23 | 30.621 | 2.9171 | 14.4 |
| 24 | 31.099 | 2.8734 | 11.6 |
| 25 | 31.722 | 2.8184 | 1.5 |
| 26 | 32.157 | 2.7812 | 1.9 |
| 27 | 33.6 | 2.6651 | 6.4 |
| 28 | 34.099 | 2.6272 | 2.4 |
| 29 | 34.259 | 2.6153 | 2.9 |
| 30 | 34.718 | 2.5818 | 3.5 |
| 31 | 35.019 | 2.5602 | 3.1 |
| 32 | 35.241 | 2.5446 | 3.3 |
| 33 | 35.583 | 2.5209 | 1 |
| 34 | 36.213 | 2.4785 | 0.8 |
| 35 | 36.618 | 2.452 | 1.7 |
| 36 | 38.163 | 2.3562 | 2.4 |
| 37 | 39.945 | 2.2551 | 1.9 |
| 38 | 40.347 | 2.2336 | 1.3 |
| 39 | 41.534 | 2.1724 | 0.9 |
| 40 | 42.241 | 2.1377 | 1.8 |
| 41 | 42.842 | 2.1091 | 4.5 |
| 42 | 43.299 | 2.0879 | 7.2 |
| 43 | 43.756 | 2.0671 | 1.2 |

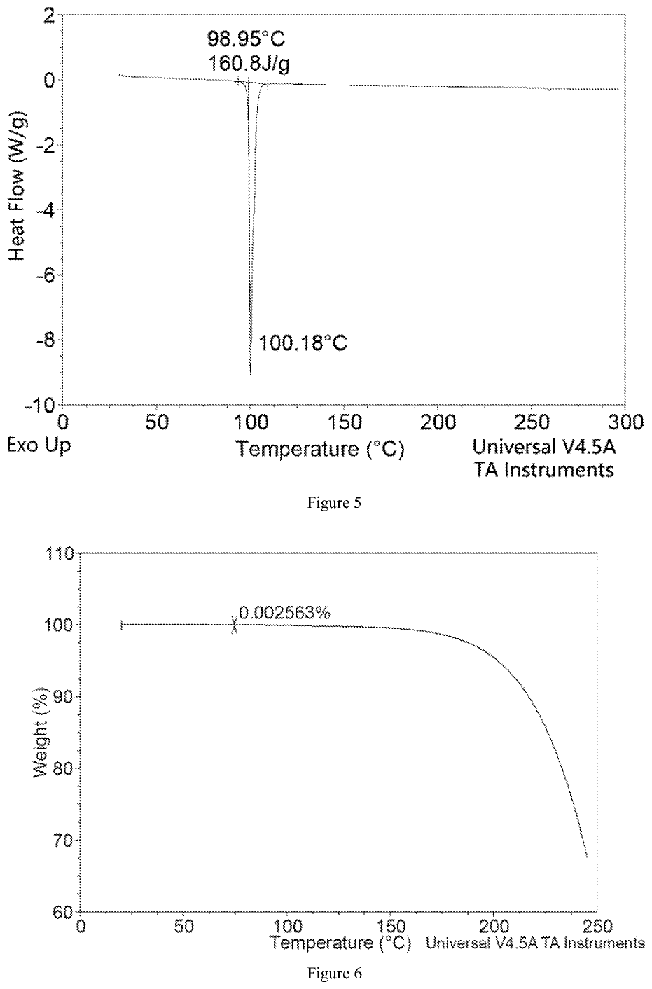

DSC and TGA curves for the white powder are measured and shown in FIGS. 5 and 6. It is observed that, the crystal form A has one characteristic endothermic peak at 96.0-107.0° C. in the DSC curve, and a weight loss of 0.002563% before 125° C. in the TGA curve, indicating the crystal form A is a non-solvate.

### Example 3 Preparation of Crystal Form A

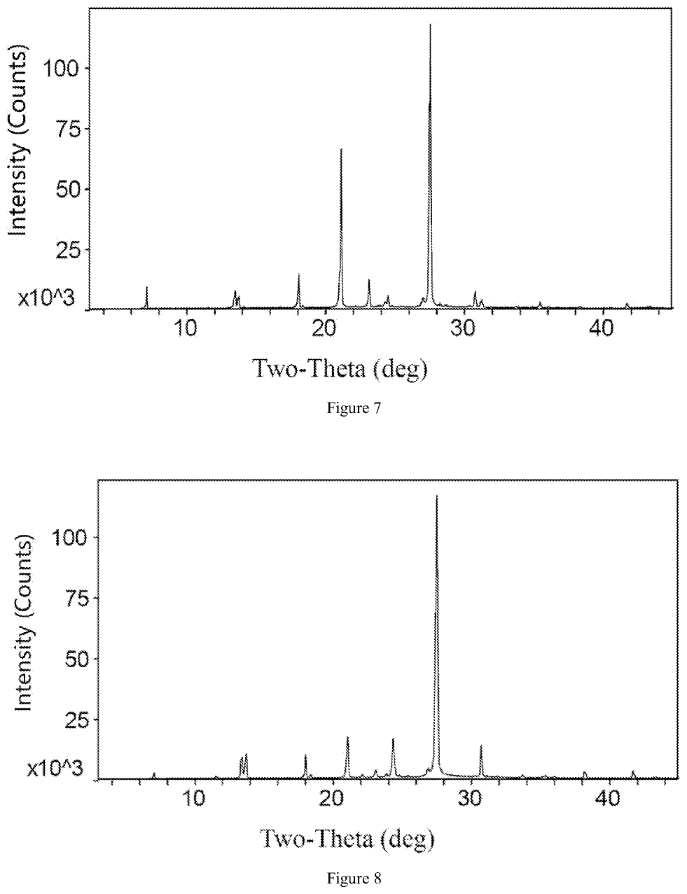

8.3 mg of the bulk pharmaceutical chemical is weighed out and placed into a sample bottle, and 0.2 mL of ethyl acetate is added thereto for dissolving at room temperature to produce a clear solution. The clear solution is allowed to undergo slow evaporation to give a solid. The solid is vacuum dried at room temperature to give a white powder. XRPD pattern is measured and shown in FIG. 7 which is substantially consistent with FIG. 4 of Example 2.

### Example 4 Preparation of Crystal Form A

8.5 mg of the bulk pharmaceutical chemical is weighed out and placed into a sample bottle, and 0.2 mL of acetone is added thereto for dissolving at room temperature to produce a clear solution. The clear solution is allowed to undergo slow evaporation to give a solid. The solid is vacuum dried at room temperature to give a white powder. XRPD pattern is measured and shown in FIG. 8 which is substantially consistent with FIG. 4 of Example 2.

### Example 5 Preparation of Crystal Form A

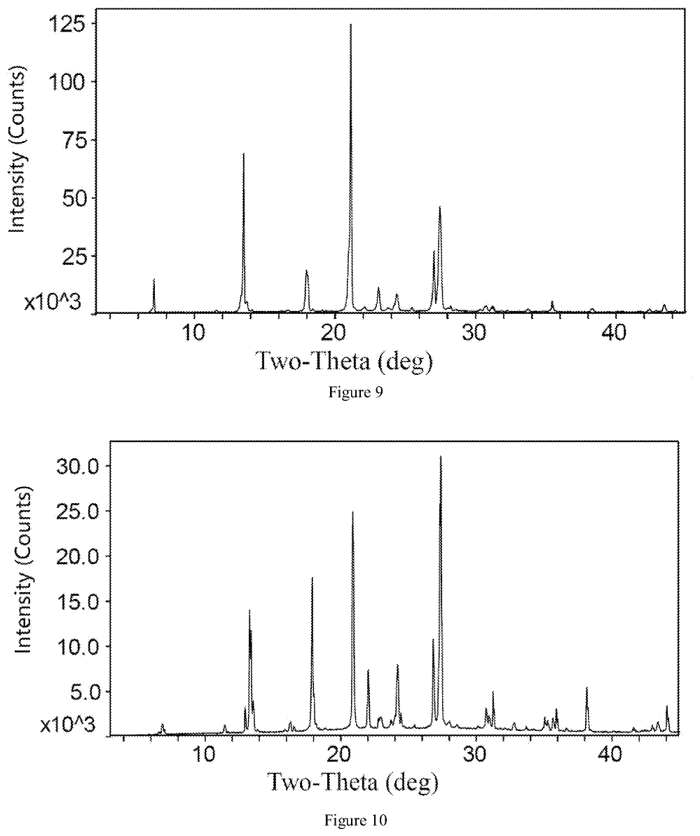

21.4 mg of the bulk pharmaceutical chemical is weighed out and placed into a sample bottle, and 0.6 mL of ethyl acetate is added thereto for dissolving at 60° C. to obtain a clear solution. The clear solution is filtered by a filter head with a diameter of 0.45 μm to obtain a filtrate, then the filtrate is cooled at 4° C. to separate a solid which is then filtered out and vacuum dried at room temperature to give a white solid. XRPD pattern of the white solid is measured and shown in FIG. 9 which is substantially consistent with FIG. 4 of Example 2.

### Example 6 Preparation of Crystal Form A

22.9 mg of the bulk pharmaceutical chemical is weighed out and placed into a sample bottle, and 0.2 mL of 4-methyl-2-pentanone is added thereto for dissolving at 60° C. to obtain a clear solution. The clear solution is filtered by a filter head with a diameter of 0.45 μm to obtain a filtrate, then the filtrate is cooled at 4° C. to separate a solid which is then filtered out and vacuum dried at room temperature to give a white solid. XRPD pattern of the white solid is measured and shown in FIG. 10 which is substantially consistent with FIG. 4 of Example 2.

### Example 7 Preparation of Crystal Form A

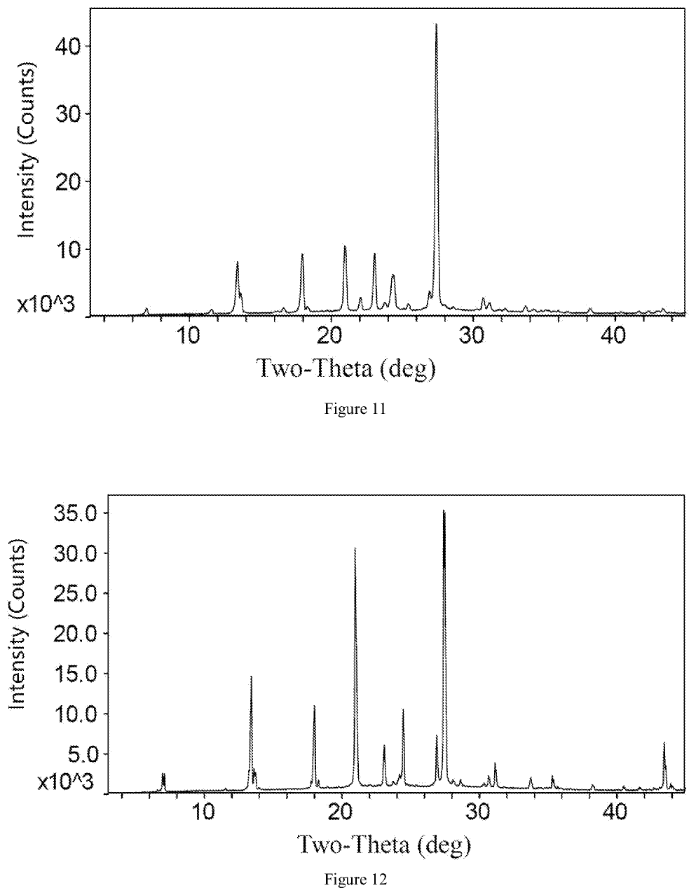

9.6 mg of the bulk pharmaceutical chemical is weighed out and placed into a sample bottle, and 0.15 mL of ethyl acetate is added for dissolving at room temperature to obtain a clear solution, and 2 mL of n-hexane is slowly added dropwise thereto to obtain a turbid solution. The turbid solution is filtered to give a solid which is then vacuum dried at room temperature to give a white solid. XRPD pattern of the white solid is measured and shown in FIG. 11 which is substantially consistent with FIG. 4 of Example 2.

### Example 8 Preparation of Crystal Form A

8.9 mg of the bulk pharmaceutical chemical is weighed out and placed into a sample bottle, and 0.6 mL of 4-methyl-2-pentanone is added for dissolving at room temperature to obtain a first clear solution, and 2 mL of absolute ethyl ether is slowly added dropwise thereto to obtain a second clear solution, followed by standing to separate a solid. The solid is collected by centrifuging, and then vacuum dried at room temperature to give a white solid. XRPD pattern of the white solid is measured and shown in FIG. 12 which is substantially consistent with FIG. 4 of Example 2.

### Experimental Example 1 Yield and Purity Study

Purities of the crystal form I in Example 1 and the crystal forms A prepared in Examples 2-8 are determined by HPLC. The results are as shown in table 3.

TABLE 3
Yield and Purity of the crystal form I
and the crystal form A

| | Yield/% | Purity/% |
|---|---|---|
| Example 1 | 85.0 | 98.6 |
| Example 2 | 96.5 | 98.9 |
| Example 3 | 97.1 | 98.8 |
| Example 4 | 98.1 | 98.9 |
| Example 5 | 88.5 | 99.2 |
| Example 6 | 86.5 | 99.5 |
| Example 7 | 90.1 | 99.6 |
| Example 8 | 90.3 | 99.5 |

### Experimental Example 2 Hygroscopicity Study

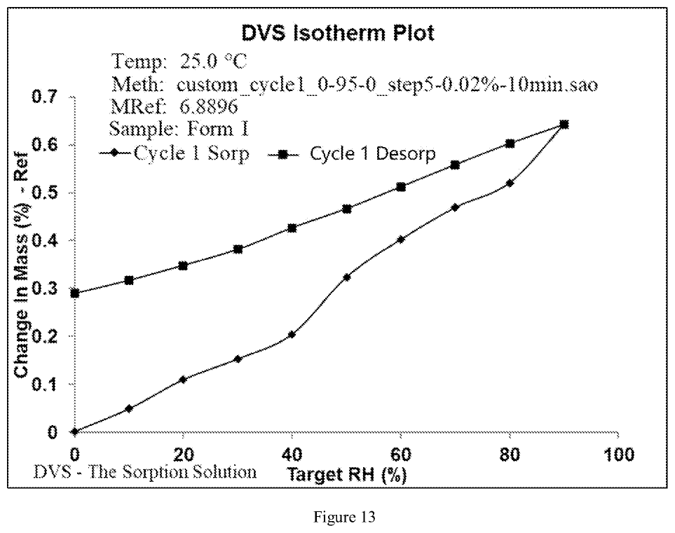

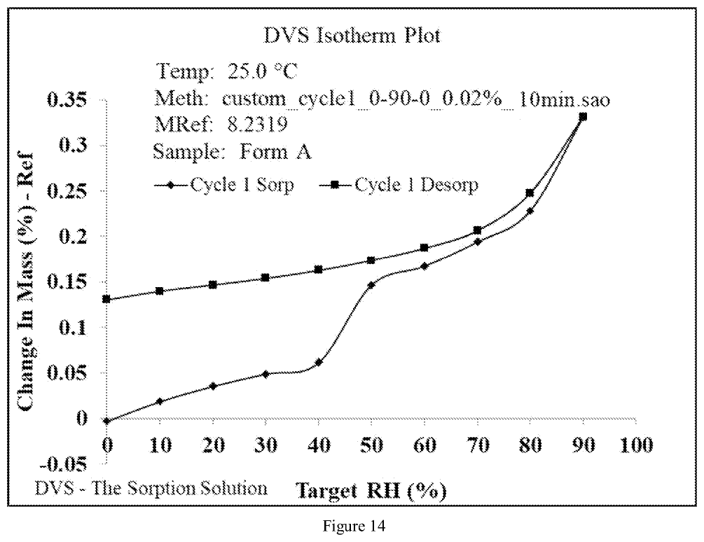

Dynamic vapor sorption (DVS) experiment are performed for the crystal form I of Example 1 and the crystal form A prepared in Example 2 to obtain DVS curves under the following conditions: the temperature is 25° C., the relative humidity (RH) is stepped up from RH 0 to RH 90% at a rate of RH 10% per step, with 10 min for each step to reach equilibrium. When RH 90% is completed, the crystal form I has a weight increase of 0.642% due to moisture absorption, as shown in FIG. 13. In contrast, when RH 90% is completed, the crystal form A has a weight increase of less than 0.35% due to moisture absorption, as shown in FIG. 14, indicating that the crystal form A has a significantly reduced hygroscopicity, which is more conducive to transportation and storage of drugs.

### Experimental Example 3 Stability Study of Crystal Form A

Following tests are performed for the crystal form A prepared in Example 2:

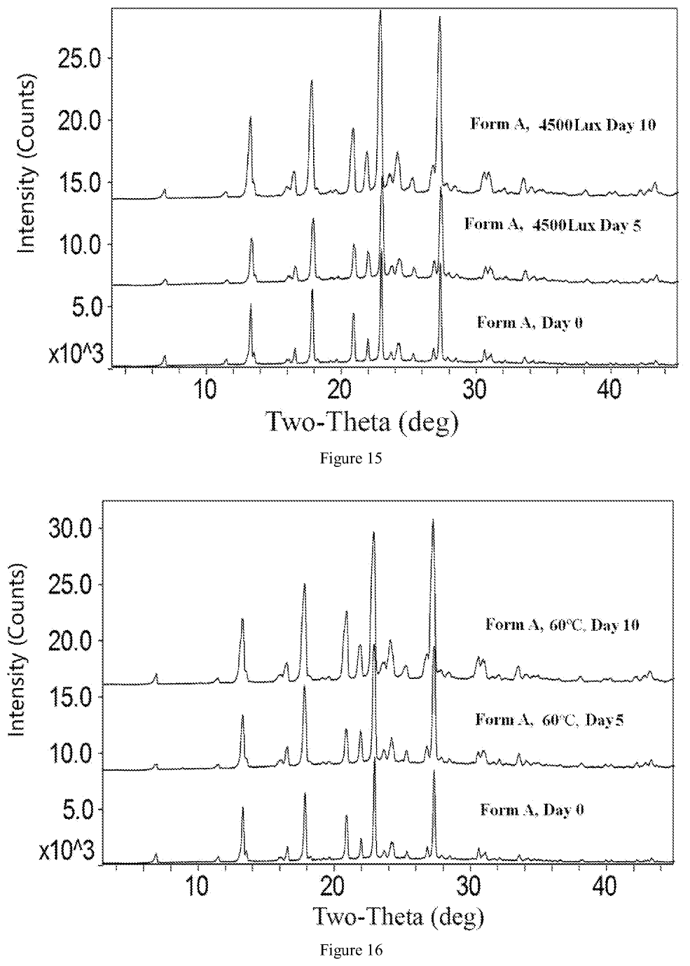

(1) Light stability: the test sample is placed in an environment having a temperature of 25° C. and a light condition of 4500 Lux for 5 days and 10 days, respectively, to test the stability of the crystal form. The results are shown in FIG. 15, indicating that the crystal form A has good light stability.

(2) High-temperature stability: the test sample is placed at a temperature of 60° C. for 5 days and 10 days, respectively, to test the stability of the crystal form. The results are shown in FIG. 16, indicating that the crystal form A has good high-temperature stability.

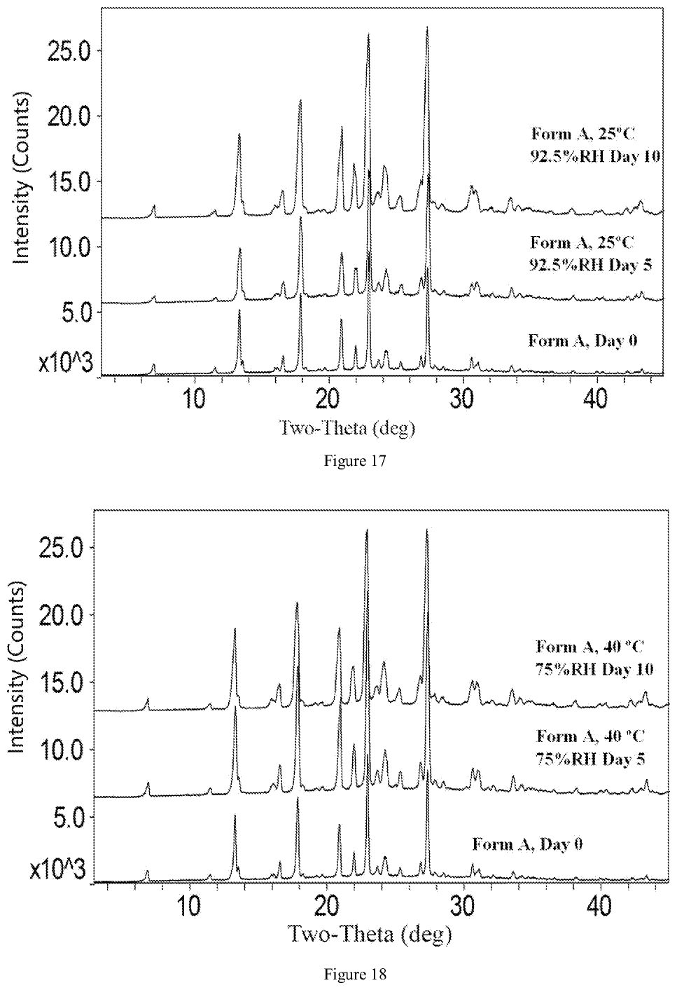

(3) High-humidity stability 1: the test sample is placed in an environment having a humidity of 92.5% RH and a temperature of 25° C. for 5 days and 10 days, respectively, to test the stability of the crystal form. The results are shown in FIG. 17, indicating that the crystal form A has good high-humidity stability.

(4) High-humidity stability 2: the test sample is placed in an environment having a humidity of 75% RH and a temperature of 40° C. for 5 days and 10 days, respectively, to test the stability of the crystal form. The results are shown in FIG. 18, indicating that the crystal form A has good high-humidity stability.

### Experimental Example 4 Stability Study of Crystal Form I

Following tests are performed for the crystal form I prepared in Example 1:

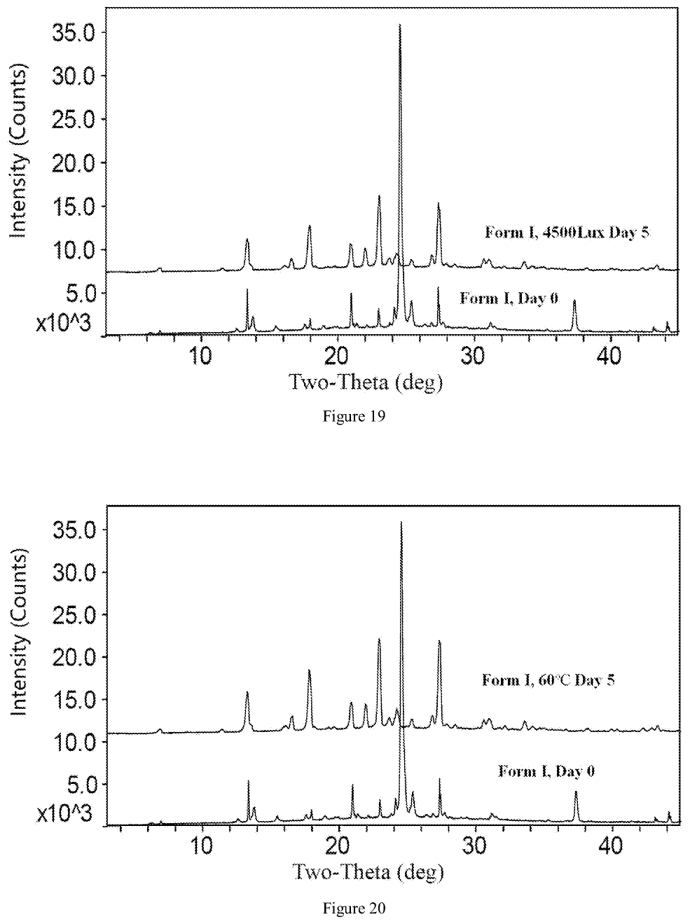

(1) Light stability: by referring to the same method of light stability test for crystal A, the test sample is placed in an environment having a temperature of 25° C. and a light condition of 4500 Lux for 5 days to test the stability of the crystal form, which is compared with XRPD pattern of the crystal form I on day 0. The results are shown in FIG. 19, indicating that the crystal form I is unstable under lights and easily transforms into crystal form A.

(2) High-temperature stability: by referring to the same method of high-temperature stability test for crystal A, the test sample is placed at a temperature of 60° C. for 5 days to test the stability of the crystal form, which is compared with XRPD pattern of the crystal form I on day 0. The results are shown in FIG. 20, indicating that the crystal form I is unstable under high temperatures and easily transforms into crystal form A.

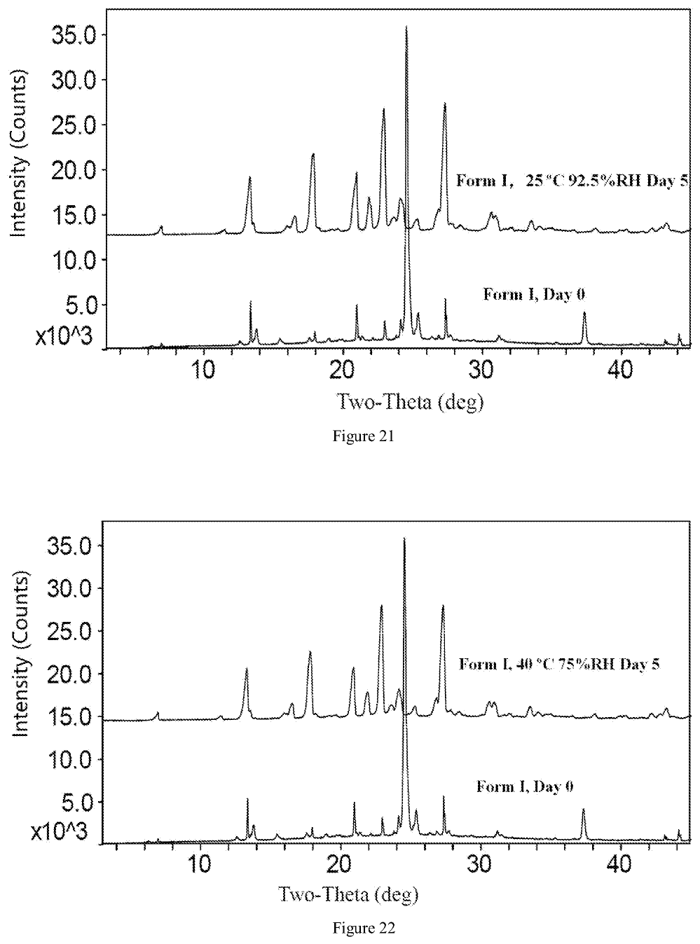

(3) High-humidity stability 1: by referring to the same method of high-humidity stability test 1 for crystal A, the test sample is placed in an environment having a humidity of 92.5% RH and a temperature of 25° C. for 5 days to test the stability of the crystal form, which are compared with XRPD pattern of the crystal form I on day 0. The results are shown in FIG. 21, indicating that the crystal form I is unstable under high humidity and easily transforms into crystal form A.

(4) High-humidity stability 2: by referring to the same method of high-humidity stability test 2 for crystal A, the test sample is placed in an environment having a humidity of 75% RH and a temperature of 40° C. for 5 days to test the stability of the crystal form, which are compared with XRPD pattern of the crystal form I on day 0. The results are shown in FIG. 22, indicating that the crystal form I is unstable under high humidity and easily transforms into crystal form A.

In summary, the crystal form A of 2-(2, 5-dioxopyrrolidin-1yl)ethyl methyl fumarate provided in the present application has good light stability, high-temperature stability and high-humidity stability. While the crystal form I is poor in light stability, high-temperature stability and high-humidity stability, and has a tendency to transform to the crystal form A of 2-(2, 5-dioxopyrrolidin-1yl)ethyl methyl fumarate of the present application.

Apparently, the aforementioned embodiments are merely examples illustrated for clearly describing the present application, rather than limiting the implementation ways thereof. For those skilled in the art, various changes and modifications in other different forms can be made on the basis of the aforementioned description. It is unnecessary and impossible to exhaustively list all the implementation ways herein. However, any obvious changes or modifications derived from the aforementioned description are intended to be embraced within the protection scope of the present application.

While embodiments incorporating the principles of the present disclosure have been disclosed hereinabove, the present disclosure is not limited to the disclosed embodiments. Instead, this application is intended to cover any variations, uses, or adaptations of the disclosure using its general principles. Further, this application is intended to cover such departures from the present disclosure as come within known or customary practice in the art to which this disclosure pertains and which fall within the limits of the appended claims.

1. A crystal form A of 2-(2, 5-dioxopyrrolidin-1yl)ethyl methyl fumarate, wherein the X-ray powder diffraction thereof using Cu-Kα radiation has characteristic peaks at 2θ diffraction angles of 13.5±0.2°, 17.9±0.2°, 23.0±0.2° and 27.3±0.2°.

2. The crystal form A of claim 1, wherein the X-ray powder diffraction thereof using Cu-Kα radiation has further characteristic peaks at 2θ diffraction angles of 13.3±0.2°, and 18.2±0.2°.

3. The crystal form A of claim 2, wherein the X-ray powder diffraction thereof using Cu-Kα radiation has further characteristic peaks at 2θ diffraction angles of 19.3±0.2°, and 19.6±0.2°.

4. The crystal form A of claim 3, wherein the X-ray powder diffraction thereof using Cu-Kα radiation has further characteristic peaks at 2θ diffraction angles of 16.6±0.2°, 20.9±0.2°, 22.0±0.2°, 24.3±0.2°, 25.3±0.2°, and 30.6±0.2°.

5. The crystal form A of claim 4, wherein the X-ray powder diffraction thereof using Cu-Kα radiation has further characteristic peaks at 2θ diffraction angles of 6.92±0.2°, 11.5±0.2°, 16.1±0.2°, 23.7±0.2°, 26.9±0.2°, and 31.1±0.2°.

6. The crystal form A of claim 1, wherein the crystal form A has following characteristic peaks in X-ray powder diffraction pattern:

| No. of Peaks | 2θ (°) | I % |
|---|---|---|
| 1 | 13.3 | 72.3 |
| 2 | 13.54 | 19.8 |
| 3 | 17.878 | 82.2 |
| 4 | 22.962 | 100 |
| 5 | 27.34 | 97.2 |

7. The crystal form A of claim 1, wherein the crystal form A has following characteristic peaks in X-ray powder diffraction pattern:

| No. of Peaks | 2θ (°) | I % |
|---|---|---|
| 1 | 6.92 | 12.8 |
| 2 | 11.481 | 6.1 |
| 3 | 13.3 | 72.3 |
| 4 | 13.54 | 19.8 |
| 5 | 16.562 | 14.5 |
| 6 | 17.878 | 82.2 |
| 7 | 18.24 | 5.1 |
| 8 | 19.282 | 2.5 |
| 9 | 19.644 | 2.9 |
| 10 | 20.901 | 53 |
| 11 | 21.98 | 18.4 |
| 12 | 22.962 | 100 |
| 13 | 24.26 | 25.7 |
| 14 | 26.858 | 13.5 |
| 15 | 27.34 | 97.2 |

8. The crystal form A of claim 1, wherein the crystal form A has following characteristic peaks in X-ray powder diffraction pattern:

| No. of Peaks | 2-Theta | I % | d (Å) |
|---|---|---|---|
| 1 | 6.92 | 12.8 | 12.764 |
| 2 | 11.481 | 6.1 | 7.7013 |
| 3 | 13.3 | 72.3 | 6.6517 |
| 4 | 13.54 | 19.8 | 6.5344 |
| 5 | 16.562 | 14.5 | 5.3481 |
| 6 | 17.878 | 82.2 | 4.9572 |
| 7 | 18.24 | 5.1 | 4.8597 |
| 8 | 19.282 | 2.5 | 4.5995 |
| 9 | 19.644 | 2.9 | 4.5155 |
| 10 | 20.901 | 53 | 4.2467 |
| 11 | 21.98 | 18.4 | 4.0405 |
| 12 | 22.962 | 100 | 3.87 |
| 13 | 23.681 | 7.3 | 3.754 |
| 14 | 24.26 | 25.7 | 3.6657 |
| 15 | 25.34 | 8.2 | 3.5119 |
| 16 | 26.858 | 13.5 | 3.3167 |
| 17 | 27.34 | 97.2 | 3.2593 |
| 18 | 30.621 | 14.4 | 2.9171 |
| 19 | 31.099 | 11.6 | 2.8734 |

9. The crystal form A of claim 1, wherein the crystal form A has an X-ray powder refraction pattern substantially as shown in FIG. 4.

10. The crystal form A of claim 1, wherein the crystal form A has a characteristic endothermic peak in a temperature range of 96.0° C.-107.0° C. measured by differential scanning calorimetry.

11. The crystal form A of claim 1, wherein the crystal form A has a differential scanning calorimetry curve substantially as shown in FIG. 5.

12. The crystal form A of claim 1, wherein the crystal form A has a weight loss of less than 0.01% before a temperature of 125° C. in its thermo gravimetric analysis curve.

13. The crystal form A of claim 1, wherein the crystal form A has a thermo gravimetric analysis curve substantially as shown in FIG. 6.

14. A method for preparing the crystal form A of claim 1, comprising the following steps of: dissolving 2-(2, 5-dioxopyrrolidin-1yl)ethyl methyl fumarate by adding a good solvent thereto, evaporating the solvent or cooling to give a solid, and drying the solid to obtain the crystal form A as a powder.

15. The method of claim 14, wherein said dissolving is performed by adding the good solvent at a temperature of 50° C. to 65° C., and said cooling is performed at a temperature of −18° C. to 4° C. to give a solid.

16. A method for preparing the crystal form A of claim 1, comprising the following steps of: dissolving 2-(2, 5-dioxopyrrolidin-1yl)ethyl methyl fumarate by adding a good solvent thereto, then adding a poor solvent, separating a solid and drying to obtain the crystal form A as a powder.

17. The method of claim 16, wherein the good solvent is added at a temperature of 15° C. to 35° C. for dissolving, and the poor solvent is added at a temperature of 15° C. to 35° C. to obtain a solid.

18. The method of claim 14, wherein the good solvent is an organic solvent selected from the group consisting of a lower alcohol, a lower ketone, a lower ester, a lower nitrile, and a lower ether;
preferably, the lower alcohol is selected from the group consisting of methanol, ethanol, isopropanol or n-butanol, the lower ketone is acetone or 4-methyl-2-pentanone, the lower ester is ethyl acetate, the lower ether is tetrahydrofuran or dioxane, and the lower nitrile is acetonitrile.

19. The method of claim 14, wherein a ratio of 2-(2, 5-dioxopyrrolidin-1yl)ethyl methyl fumarate to the good solvent is (10-40) mg:(0.1-5) mL.

20. The method of claim 16, wherein the poor solvent is select from n-heptane, n-hexane, or absolute ethyl ether.

21. A pharmaceutical composition, comprising the crystal form A of claim 1 and a pharmaceutically acceptable excipient.

22. A method for treating a neurological disease, comprising administering a pharmaceutically effective amount of the crystal form A of claim 1 or a pharmaceutical composition comprising the same.

23. The method of claim 22, wherein the neurological disease is multiple sclerosis or psoriasis.

24. The method of claim 22, wherein the medicament is administered orally, parenterally, intradermally, intrathecally, intramuscularly, subcutaneously, vaginally, as a buccal, sublingually rectally, as a topical, inhalation, intranasal, or transdermally.
